(19) United States
(12) Patent Application Publication
(10) Pub. No.: US 2008/0312655 A1
(43) Pub. Date: Dec. 18, 2008

(54) POLYAXIAL SCREW SYSTEM AND METHOD HAVING A HINGED RECEIVER

(75) Inventors: DAVID LOUIS KIRSCHMAN, DAYTON, OH (US); BRIAN J. HEMMELGARN, DAYTON, OH (US)

Correspondence Address: MATTHEW R. JENKINS, ESQ., 2310 FAR HILLS BUILDING, DAYTON, OH, 45419, US

(73) Assignee: X-SPINE SYSTEMS, INC., MIAMISBURG, OH (US)

(21) Appl. No.: 11/762,911

(22) Filed: Jun. 14, 2007

(51) Int. Cl. A61B 17/04; A61B 17/70

(52) U.S. Cl. 606/60; 606/301; 606/305; 606/264

(57) ABSTRACT

A polyaxial screw system and method are provided. The system comprises a receiver having a pivot area defined by a pivot joint or a material between a pair of receiver walls or members that permit the receiver walls or members to move or pivot relative to each other so that a dimension of a socket area can be changed. The socket area is defined by socket walls that when at least one of the receiver members pivots relative to another receiver member or wall, a distance between the socket walls gets smaller so that the socket walls can engage and lock against a head of a polyaxial screw.

### BACKGROUND OF THE INVENTION

1. Field of the Invention

This invention relates to a polyaxial screw system and method comprising a hinged receiver.

2. Description of the Related Art

Various methods of spinal immobilization have been known and used in the past. The preferred treatment for spinal stabilization is immobilization of the joint by surgical fusion or anthrodesis. This method has been known since development in 1911 by Hibbs and Albe. However, in many cases, in particular cases involving fusion across the lumbosacral articulation and where there are many levels involved, pseudorarthrosis is a problem. It was discovered that immediate immobilization was necessary in order to allow a bony union to form. Post operative external immobilization, such as the use of splints and casts, was a favored method of treatment, however, as surgical techniques have become more sophisticated, various methods of internal and external fixation have been developed.

Internal fixation refers to therapeutic methods of stabilization which are wholly internal to the patient and include commonly known devices such as bone plates and pins. External fixation, in contrast, involves at least some portion of stabilization device which is external to the patient's body. Internal fixation is now the favored method of immobilization because the patient is allowed greater freedom with the elimination of the external portion of the device and the possibility of infection, such as a pin tract infection is reduced.

There have been numerous systems and methods developed in the past for correcting and stabilizing and aligning the spine for facilitating, for example, fusion at various levels or areas of the spine, such as those devices are shown in U.S. Pat. Nos. 4,085,744; 4,269,178; 4,805,602; 5,466,237; 5,474,555; 5,891,145; and 6,869,433 B2. Bone screws with a polyaxial head are commonly used in spine surgery today. They are used chiefly in the lumbar spine and screwed into bone (pedicle) posteriorly. The head of the screw is attached to the shaft of the screw by means of a ball and socket. The top of the screw is machined into a ball, and the head contains a socket into which the ball fits. The screw head further contains a receiver for receiving a separate rod. The rod is fastened to the screw head receiver via a threaded cap. The rod is then fastened to screws placed in adjacent vertebrae thus providing stabilization. The polyaxial head allows the rod to be placed in a variety of angles with respect to the screw allowing conformance to local anatomy.

When the threaded cap is tightened upon the rod, a frictional pressure is transmitted from the threaded cap to the rod thence to the top of the ball, thus locking the ball-in-socket and preventing motion after tightening has occurred. This concept is demonstrated in U.S. Pat. Nos. 5,466,237 and 5,474,555, which illustrate this type of screw.

U.S. Pat. No. 5,466,237 to Bird et al. discloses a bone screw having a spherical projection on the top of the bone screw. An externally threaded receiver member supports the bone screw and spinal rod on top of the spherical projection. An outer nut is tightened onto the receiver member to press the spinal rod against the spherical projection to accommodate various angular orientations of the bone screw relative to the rod.

In another approach shown in U.S. Pat. No. 4,946,458 to Harms, a spherical headed bone screw supported within separate halves of a receiving member. The bottoms of the halves are held together by a retaining ring. The top of the receiver halves are compressed about the bone screw by nuts threaded onto a threaded spinal rod.

In still another approach taken by Harms et al. in U.S. Pat. No. 5,207,678, a receiver member is flexibly connected about a partially spherical head of a bone screw. Conical nuts on opposite sides of the receiver member are threaded onto a threaded rod passing through the receiver. As the conical nuts are threaded toward each other, the receiver member flexibly compresses around the head of the bone screw to clamp the bone screw in its variable angular position. One detriment of the systems in the two Harms et al. patents is that the spinal rod must be threaded in order to accept the compression nuts.

U.S. Pat. No. 6,869,433 discloses the use of a pedicle screw assembly that comprises a screw having a head with a convex portion and a receiver that receives the head. The receiver also receives an elongated member, such as a spinal fixation rod. The receiver has a concave portion which has a radius of curvature which is less than the radius of curvature of the convex portion of the head whereby to create an interference fit between the convex portion of the head and the concave portion of the receiver. The device also includes an internal nut and external nut that compresses the rod against a pressure disc which in turn compresses the head convex portion of the screw into the receiver concave portion and locks the angular position of the receiver with respect to the screw.

Many of the receivers of the past required the screw to be top-loaded or inserted through a rod-receiving area and through a hole in a bottom of the receiver. A compression member sometimes was situated in the receiver and then means for forcing the compression member to engage the polyaxial screw and force it against a bottom wall of the receiver was one approach in the past.

What is needed, therefore, is a system and method that provides improved fixation and locking between an elongated member or rod and a screw.

### SUMMARY OF THE INVENTION

The present invention provides an improved fixation and locking system and method between an elongated member or rod and a screw that is screwed into bone.

One object of the invention is to provide a system and method for permitting bottom-loading of a screw into a receiver.

Another object of the invention is to provide a simple locking connection between a screw and a socket of a receiver.

Still another object of the invention is to provide a receiver adapted to change a dimension of a socket or area so that the socket can engage and lock against a head of a screw.

Still another object of the invention is to provide a receiver having a pivot area.

Another object of the invention is to provide a receiver having a pivot area that permits at least a portion of a receiver to pivot so as to enable a socket wall to engage and lock against a head of a polyaxial screw.

In one aspect, one embodiment comprises a polyaxial screw system comprising a receiver comprising a socket for receiving a head of a polyaxial screw and a rod-receiving area for receiving a rod, the receiver having a pivot area to permit a dimension of the socket to be changed in order to apply the locking force onto a head of the polyaxial screw.

In another aspect, one embodiment comprises a bone fixation system comprising a receiver comprising a socket defining a socket area, the socket comprising at least one socket wall, the socket being adapted to receive a screw head of a polyaxial screw, a rod-receiving area defined by at least one rod-receiving wall, the rod-receiving area being adapted to receive a rod, and a pivot area between the rod-receiving area and the socket area, the pivot area permitting a dimension of the rod-receiving area to increase and substantially simultaneously being adapted to permit a dimension of the socket area to decrease so that the at least one socket wall may become locked to the screw head in response to movement of the at least one rod-receiving wall.

In still another aspect, one embodiment comprises a receiver comprising a first rod-receiving wall and generally opposed second rod-receiving wall, the first and second rod-receiving walls cooperating to define a rod-receiving area for receiving a rod, a first socket wall and a generally opposed second socket wall, the first and second socket walls cooperating to define a socket area for receiving a screw head of a screw and an intermediate area between the rod-receiving area and the socket area for permitting the first and second socket walls to move toward and away from each other in response to movement of at least one of the first or second rod-receiving walls.

In yet another aspect, one embodiment comprises a method for securing a polyaxial screw to a rod, the method comprising the steps of providing a receiver comprising a socket area for receiving a head of a polyaxial screw and a rod-receiving area for receiving a rod; and enabling the receiver to pivot or flex at an intermediate area so that when a dimension of the rod-receiving area gets larger, a dimension of the socket area substantially simultaneously gets smaller to clamp against a screw head of the screw.

In still another aspect, one embodiment comprises a method of assembling a receiver comprising the steps of providing a receiver having a pivot area, activating the receiver to increase a dimension of a socket area, inserting a screw head into the socket area; and activating the receiver to capture the screw head in the socket area.

These and other objects and advantages of the invention will be apparent from the following description, the accompanying drawings and the appended claims.

### BRIEF DESCRIPTION OF THE DRAWINGS

FIG. 1 is a view of a receiver with pivot connection in accordance with one embodiment of the invention;

FIG. 2 is an exploded view of the embodiment shown in FIG. 1;

FIG. 3 is a view of the embodiment shown in FIG. 1 before a cap is received in the receiver;

FIG. 4 is a sectional view of the assembled receiver after it has received a rod;

FIG. 5 is another view similar to FIG. 3 after the cap has been received in the receiver illustrating the cap urging the receiver members away from each other in order to change a dimension of the socket areas so that the socket walls may engage and lock against the head of the polyaxial screw;

FIG. 6 is a sectional view of the illustration shown in FIG. 5;

FIG. 7 illustrates the two receiving members that comprise the receiver;

FIG. 8 is an illustration showing the first and second receiving members pivotally coupled;

FIG. 9 is an illustration showing the receiving members pivoted so that a socket area can receive a head of a polyaxial screw;

FIG. 10 is a view of the receiver after a socket area defined by the receiver receives the head of the screw;

FIG. 11 illustrates an exploded view of the receiver after it receives the screw but before it receives a rod;

FIG. 12 is an assembled view illustrating a cap threadably received in the receiver and after the rod has been locked onto the head of the polyaxial screw;

FIG. 12A is a top plan view taken along line 12A-12A of FIG. 10;

FIG. 13 is a view of another embodiment of the invention that utilizes a guide sleeve;

FIG. 14A is an exploded view of the embodiment illustrated in FIG. 13;

FIG. 14B is a view of one of the receiver members illustrating various details of the internal walls thereof;

FIG. 14C is an illustration of a guide sleeve in accordance with the illustration;

FIG. 15 is a view of a receiver after it has received a guide sleeve but before a cap is rotatably received in the rod-receiving area;

FIG. 16 is a sectional view of the illustration shown in FIG. 15;

FIG. 17 is a view of the receiver after the cap has been received in the receiver illustrating the cap urging the receiver members away from each other in order to change a dimension of the socket areas so that the socket walls may engage and lock against the head of the polyaxial screw;

FIG. 18 is a sectional view of the illustration shown in FIG. 17;

FIG. 19 illustrates the two receiving members that comprise the receiver;

FIG. 20 is an illustration showing the first and second receiving members pivotally coupled;

FIG. 21 is an illustration showing the receiving members pivoted so that a socket area can receive a head of the polyaxial screw;

FIG. 22 is a view of the receiver after the socket area defined by the receiver receives the screw head;

FIG. 23 illustrates an exploded view of the receiver after it receives the screw, but before it receives the guide sleeve and rod;

FIG. 24 is an assembled view illustrating the cap, rod and guide sleeve received in the receiver and after the rod has been locked onto the head of the polyaxial screw;

FIG. 24A, which is on the same sheet as FIGS. 12A and 36A, is a top plan view taken along line 24A-24A of FIG. 23, with portions in cross-section;

FIG. 25 is a view of still another illustration showing the use of a compression member with the receiver;

FIG. 26A is an exploded view of the illustration shown in FIG. 25;

FIG. 26B is a view of one of the receiver members;

FIG. 26C is a view of a compression member in accordance with the illustration being described;

FIG. 27 is a view showing the compression member received in the receiver and before the cap has been secured to the receiver;

FIG. 28 is a sectional view of the illustration shown in FIG. 27;

FIG. 29 is a view of the receiver after the cap has been received in the receiver illustrating the cap urging the receiver members away from each other in order to change a dimension of the socket areas so that the socket walls may engage and lock against the head of the polyaxial screw;

FIG. 30 is a sectional view of the illustration in FIG. 29;

FIG. 31 illustrates the two receiving members that comprise the receiver;

FIG. 32 is an illustration showing the first and second receiving members pivotally coupled;

FIG. 33 is an illustration showing the receiving members pivoted so that a socket area can receive a head of a polyaxial screw;

FIG. 34 is a view of the receiver after a socket area defined by the receiver receives the head;

FIG. 35 illustrates an exploded view of the receiver after it receives the screw and with the compression member and rod in exploded view;

FIG. 36 is an assembled view illustrating the cap, rod and compression member received in the receiver and after the rod has been locked onto the head of the polyaxial screw;

FIG. 36A, which is on the same sheet at FIGS. 12A and 24A, is a top plan view taken along line 36A-36A of FIG. 35, with portions in cross-section;

FIG. 37 is a fragmentary view of the receiver showing a cap that has a plurality of male projections that are received in generally L-shaped female channels to provide a bayonet connection;

FIG. 38 is a view illustrating a view after the cap has been received in the receiver and before it is rotated to a locked position;

FIG. 39 is a sectional view taken along the line 39-39 in FIG. 38 plan view of the illustration shown in FIG. 38;

FIG. 40 is a sectional view taken along the line 40-40 in FIG. 38 illustrating the cap when it is in the down, but unlocked, position;

FIG. 41 is a sectional view illustrating the cap after it has been rotated to the locked position;

FIG. 42 illustrates the cooperation of the male member in the generally L-shaped female channel when the cap has been rotated to the locked position;

FIG. 43 is a sectional view taken along the line 43-43 in FIG. 41 showing the cap after it has been rotated to the locked poison;

FIG. 44 is another illustration showing an integrally formed pivot connection in another illustration;

FIG. 45 is an exploded view of the illustration shown in FIG. 44;

FIG. 46 is a view illustrating the receiver walls pivotally connected with relief areas to permit such pivoting; and

FIG. 47 is a view illustrating the pivotal movement of the receiver walls or members after they have pivoted in order to change and make smaller a socket area so that the socket wall can engage and lock against the head of the polyaxial screw.

### DETAILED DESCRIPTION OF PREFERRED EMBODIMENTS

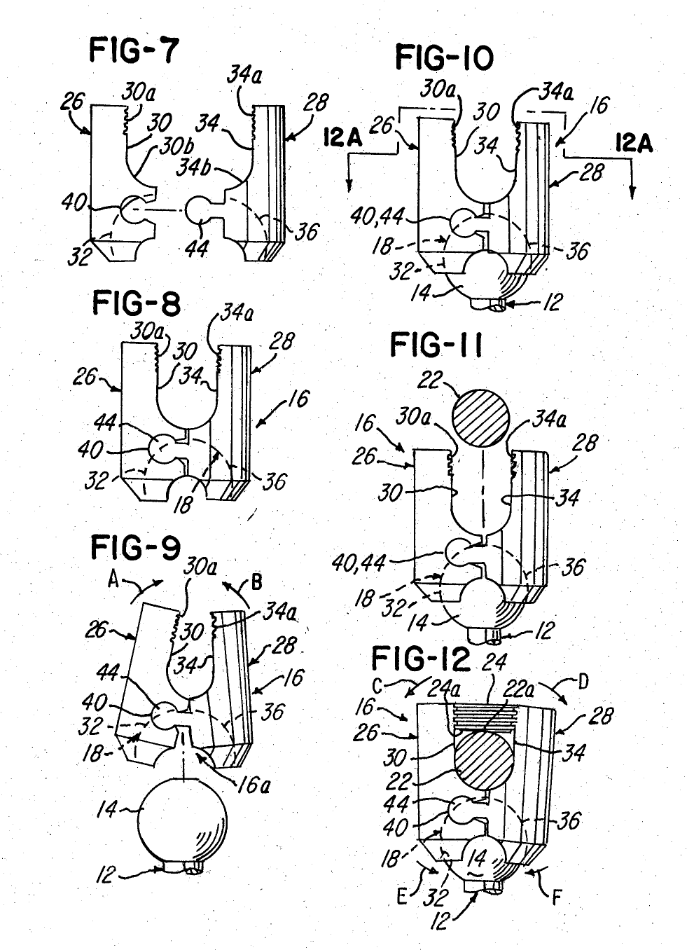

Referring now to FIG. 1, a polyaxial screw system 10 is shown comprising a screw 12 having a polyaxial screw head 14. The polyaxial screw system 10 further comprises a receiver 16 adapted to comprise or define a socket area 18 (FIG. 4) for receiving the polyaxial screw head 14 of the screw 12. The receiver 16 is further adapted to comprise or define a rod-receiving area 20 for receiving a rod 22 as illustrated in FIGS. 1-12.

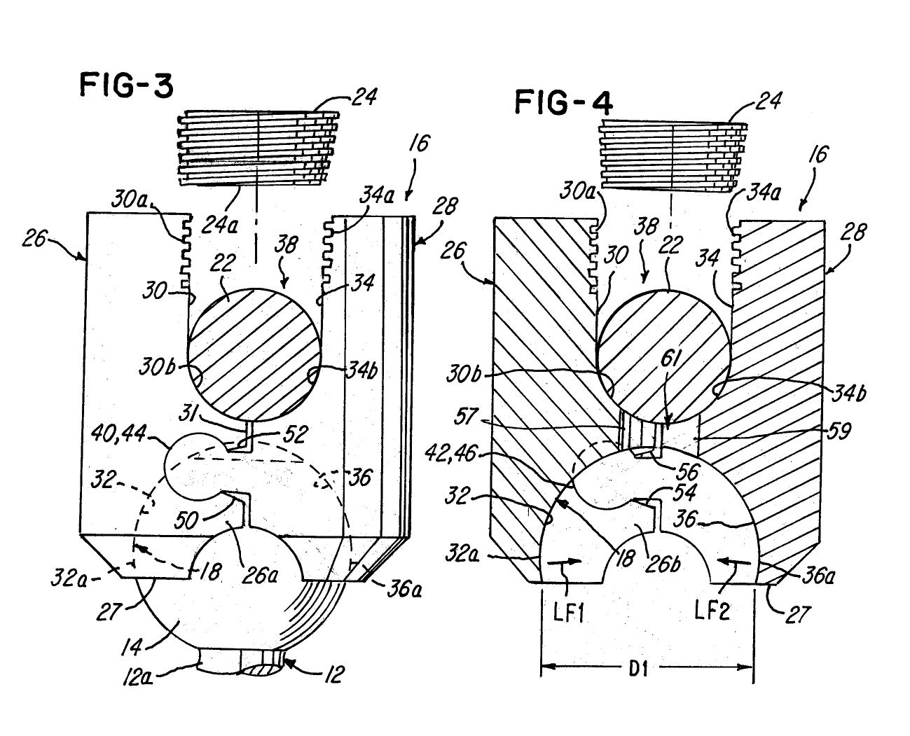

The receiver 16 in this embodiment comprises a first receiver member 26 and a second receiver member 28. As illustrated in FIGS. 3 and 4, the first receiver member 26 comprises a first rod-receiving wall 30 and a first socket wall 32 and an intermediate area 31 between the walls 30 and 32. The second receiver member 28 comprises a second rod-receiving wall 34 and a second socket wall 36 and a second intermediate area 35 between the walls 34 and 36. The first and second receiver members 26 and 28 are pivotally coupled at the intermediate areas 31 and 35 so that the first rod-receiving wall 30 and first socket wall 32 become generally opposed to the second rod-receiving wall 34 and second socket wall 36, respectively.

The first rod-receiving wall 30 and second rod-receiving wall 34 comprise a first plurality of threads 30a and a second plurality of threads 34a, respectively, which cooperate to provide a threaded opening for receiving a threaded cap or screw 24 in this embodiment.

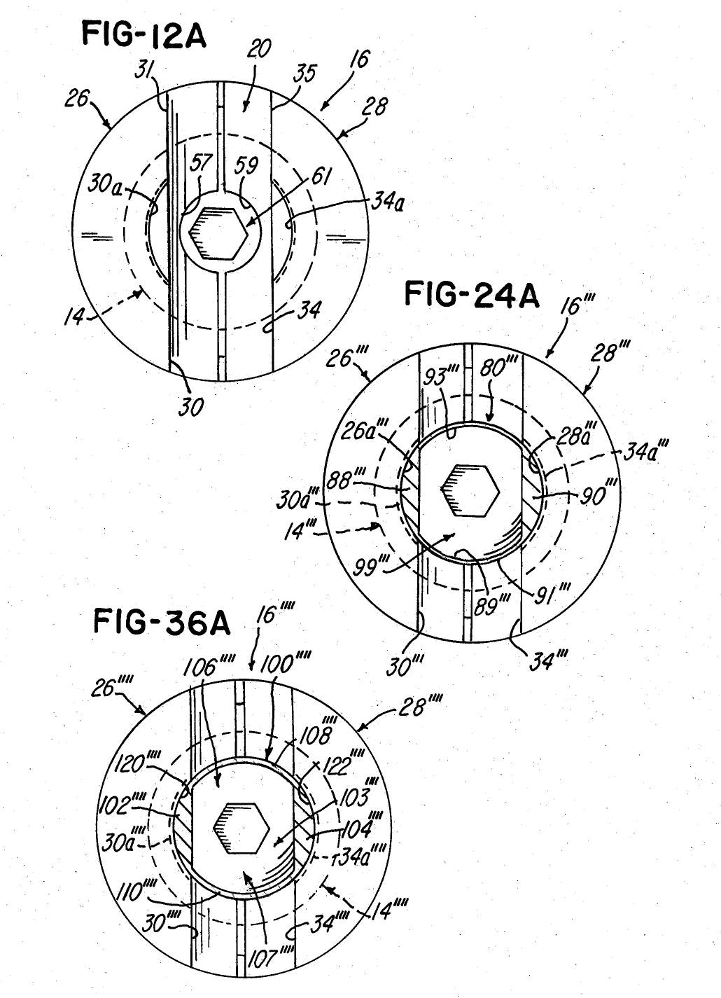

The first rod-receiving wall 30 comprises a curved area 30b and second rod-receiving wall 34 comprises a curved area 34b that cooperate to provide a generally U-shaped channel or rod-receiving area 38 that extends radially relative to an axis of the receiver 16. At a bottom of the rod-receiving area 38 (FIGS. 3 and 4) the first and second receiver members 26 and 28 comprise walls 57 and 59 that cooperate to define an area 61. The area 61 provides an aperture through which a tool (not shown) may be inserted into the female opening of the polyaxial screw head 14 in order to rotate the screw. This shown in FIG. 12A. The generally U-shaped channel 38 generally complements a shape or circumference of the rod 22 to support the rod 22 as shown. Likewise, note that the first socket wall 32 and second socket wall 36 are adapted, configured, dimensioned and cooperate to define the socket area 18 and to generally complement the spherical shape of the polyaxial screw head 14, as illustrated in FIGS. 3, 5 and 6. The socket walls 32 and 36 are dimensioned to permit relative angular movement of the receiver 16 on the polyaxial screw head 14 during an unlocked position, but also lock onto the polyaxial screw head 14 when the socket walls 32 and 36 are moved to a locked position described later herein.

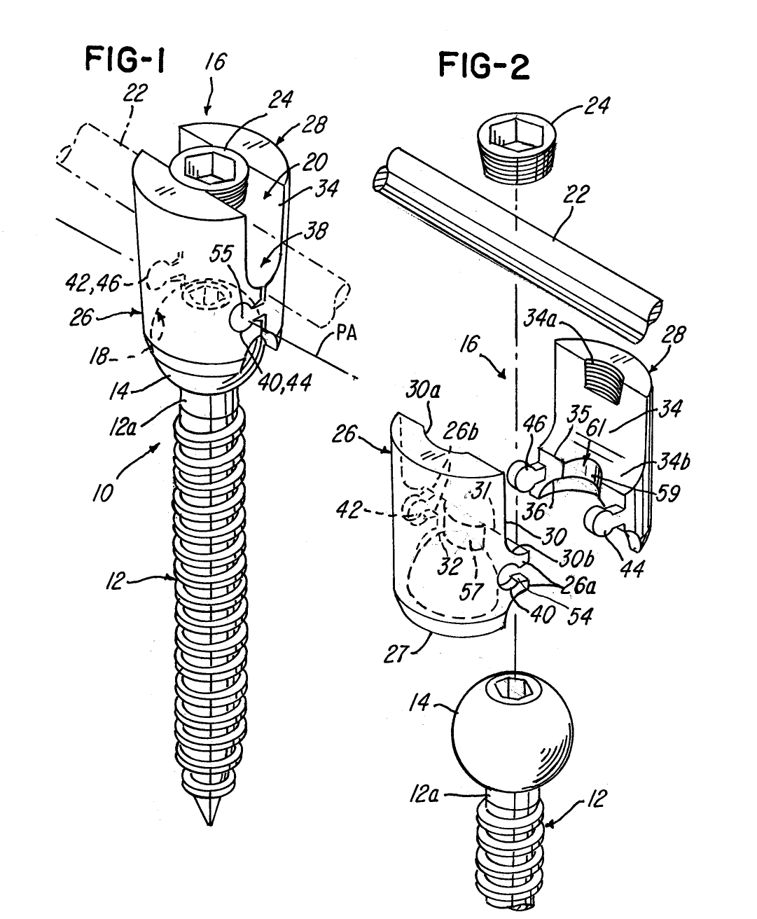

As shown in FIG. 2, the first receiver member 26 comprises a first wall projection or member 26a and a second wall projection or member 26b that define a first female recess or opening 40 and a second female recess or opening 42, respectively, as shown. The second receiver member 28 comprises a plurality of male projections 44 and 46 that are received in the first and second recesses or openings 40 and 42, respectively, as illustrated in FIGS. 1 and 2. The male projections 44 and 46 are received in the openings 40 and 42 and define, permit or provide a pivot connection about a pivot axis PA (FIG. 1) at a pivot area 55 (FIG. 1). Advantageously, the pivot area 55 and pivoting connection permits the first and second receiver members 26 and 28 to toggle or pivot relative to each other to permit a dimension of both the rod-receiving area 38 and socket area 18 to be changed.

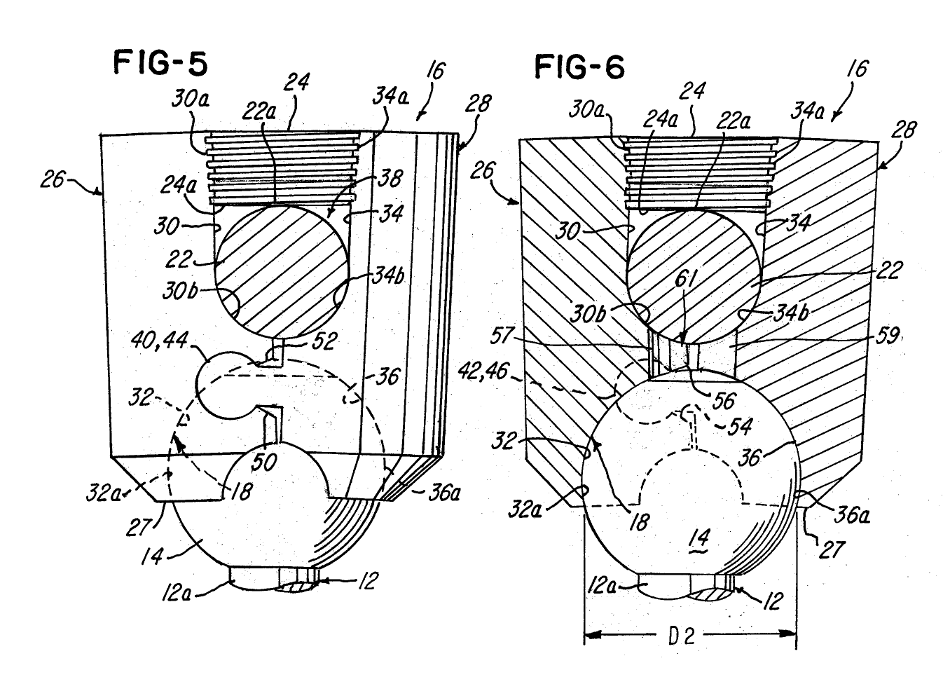

As described in more detail later herein relative to FIGS. 5-12, the pivot area 55 enables the socket walls 32 and 36 to move toward each other to apply a locking force to the polyaxial screw head 14 of the screw 12 in order to lock the rod 22 to the receiver 16. Notice that the first wall projection or member 26a (FIG. 1) comprises a plurality of relief or chamfer surface areas 50 (FIG. 3) and 52 and the second wall projection or member 26b comprises the chamfer areas 54 (FIG. 4) and 56. The relief or chamfer surface areas 50-56 permit or enable the first and second receiver members 26 and 28 to move relative to each other or pivot about the pivot axis PA of the male projections 44 and 46 and with respect to each other.

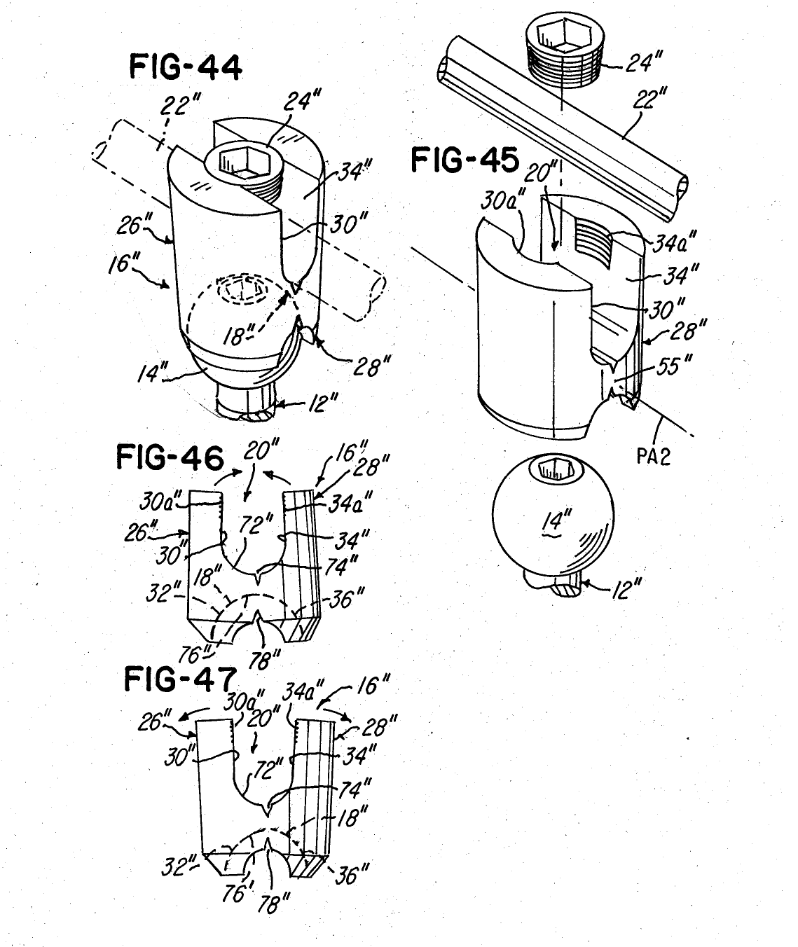

Notice that the pivot area 55 is situated between the rod-receiving area 38 and the socket area 18. In the illustration being described, the pivot axis PA is coaxial with an axis of the male projections 44 and 46 after they are received in their respective female openings 40 and 42. However, the first receiver member 26 and second receiver member 28 may be coupled together by other means or integrally formed as described later herein relative to FIGS. 44-47, while maintaining the pivot area 55 between the rod-receiving area 38 and socket area 18. Thus, a feature of the embodiment being described is that the first and second receiver members 26 and 28 are permitted to teeter or pivot about pivot axis PA between the rod-receiving area 20 and the socket area 18. This movement enables a dimension of the socket area 18 to be changed and further enables the socket walls 32 and 36 to move toward, engage and lock against the polyaxial screw head 14 of the screw 12 after the polyaxial screw head 14 is received in the socket area 18. In another embodiment, the first and second receiver members 26 and 28 are integrally formed or coupled at the pivot area 55, and that illustration in FIGS. 44-47 will be described later herein.

A method for locking the rod 22 to the polyaxial screw head 14 of the screw 12 will now be described relative to FIGS. 5-12. In this embodiment, the male projection members 44 and 46 are slid into and received by the openings 40 and 42, respectively, as illustrated in FIGS. 1 and 8, so that the first and second receiver members 26 and 28 are assembled and cooperate to define the receiver 16.

As illustrated in FIG. 9, the first and second receiver members 26 and 28 are pivoted in the direction of arrows A and B relative to each other in order to increase a dimension of the socket area 18 as shown. As illustrated in FIGS. 9-10, the polyaxial screw head 14 of screw 12 is bottom-loaded into the socket area 18, and the first and second receiver members 26 and 28 are pivoted in the directions opposite of arrow A and B relative to each other to the position shown in FIG. 10. Note in FIG. 4 and the plan view in FIG. 12A that the first receiver member 26 comprises an arcuate or curved wall 57 and second receiver member 28 comprises a curved wall 59 that cooperate to define an opening 61 through with a tool may be inserted to screw the screw 12 (FIG. 2). The polyaxial screw head 14 is now captured in the socket area 18 (FIGS. 8 and 9). Thereafter or during a surgery, for example, the rod 22 is received in the rod-receiving area 20 between the first and second receiver members 26 and 28, as illustrated in FIGS. 11 and 12. The cap or screw 24 is then threaded into the threads 30a and 34a.

Note that in the illustration being described, the cap or screw 24 is generally trapezoidal in cross-section so that as it is screwed into the receiver 16, the rod-receiving walls 30 and 34 are urged or forced away from each other. The cap or screw 24 is screwed until a bottom 24a engages the surface 22a of the rod 22, as illustrated in FIGS. 5, 6 and 12. In response to the movement of the first and second rod-receiving walls 30 and 34 in the direction of arrow C and C in FIG. 12, the first and second socket walls 32 and 36 (FIG. 4) are urged toward each other in the direction of arrows E and F (FIG. 12) until the socket walls 32 and 36 engage the polyaxial screw head 14 of the screw 12. It should be understood that the first and second socket walls 32 and 36 engage the polyaxial screw head 14 with a locking force so that the receiver 16 becomes locked onto the polyaxial screw head 14, thereby locking the rod 22 to the screw 12. Note also that the bottom 24a of the cap or screw 24 engages the rod 22 with a compressive force and forces it against the curved areas 30b and 34b (FIG. 5) to provide further locking of the rod 22 into the receiver 16, as illustrated in FIGS. 5, 6 and 12.

Advantageously, the receiver 16 (FIG. 1) is adapted to permit the first and second receiver members 26 and 28 to move or pivot with respect to each other so that a dimension, such as dimension D1 in FIG. 4, may be changed. As mentioned earlier herein, when the first socket wall 32 and second socket wall 36 move toward each other, they lockingly engage the polyaxial screw head 14 of the screw 12. Notice in FIGS. 5 and 6 that the rod 22 does not directly engage the polyaxial screw head 14 of the screw 12 in the illustration being described.

Thus it should be understood that when the threaded cap 24 is threadably received between the threads 30a and 34a, a lateral force is applied outwardly to the rod-receiving walls 30 and 34 (FIG. 4) to urge them away from each other and away from a longitudinal axis of the receiver 16. This movement in turn causes the first socket wall 32 and the second socket wall 36 to apply a lateral or radial force, relative to the receiver axis and in the direction of arrows LF1 and LF2, respectively, as shown in FIG. 4. The lateral force is enough force to provide the locking force for locking the receiver 16 onto the polyaxial screw head 14 of screw 12. It should be understood that when the first and second socket walls 32 and 36 are in the locked position and engage the polyaxial screw head 14 of screw 12, the dimension D2 of the opening between edges 32a and 36a (FIG. 6) is smaller than a diameter of the polyaxial screw head 14, which prevents the polyaxial screw head 14 from exiting the socket area 18. As mentioned earlier and as illustrated in FIGS. 9 and 10, the polyaxial screw head 14 is inserted into the socket area 18 from a bottom 16a of the receiver 16 to provide a receiver 16 that is bottom-loaded.

Notice that the first receiver member 26 has a bottom surface 27 that does not extend to the shank 12a (FIG. 3) of screw 12 or beyond a bottom edge 32a of wall 32 or edge 36a of second socket wall 36. This feature permits and facilitates angulations of the receiver 16 relative to the screw 12 in any desired polyaxial direction. In other words, the receiver 16 may be angulated relative to the polyaxial screw head 14 so that the angle of its axis relative to an axis of the screw may be angulated during a surgical procedure.

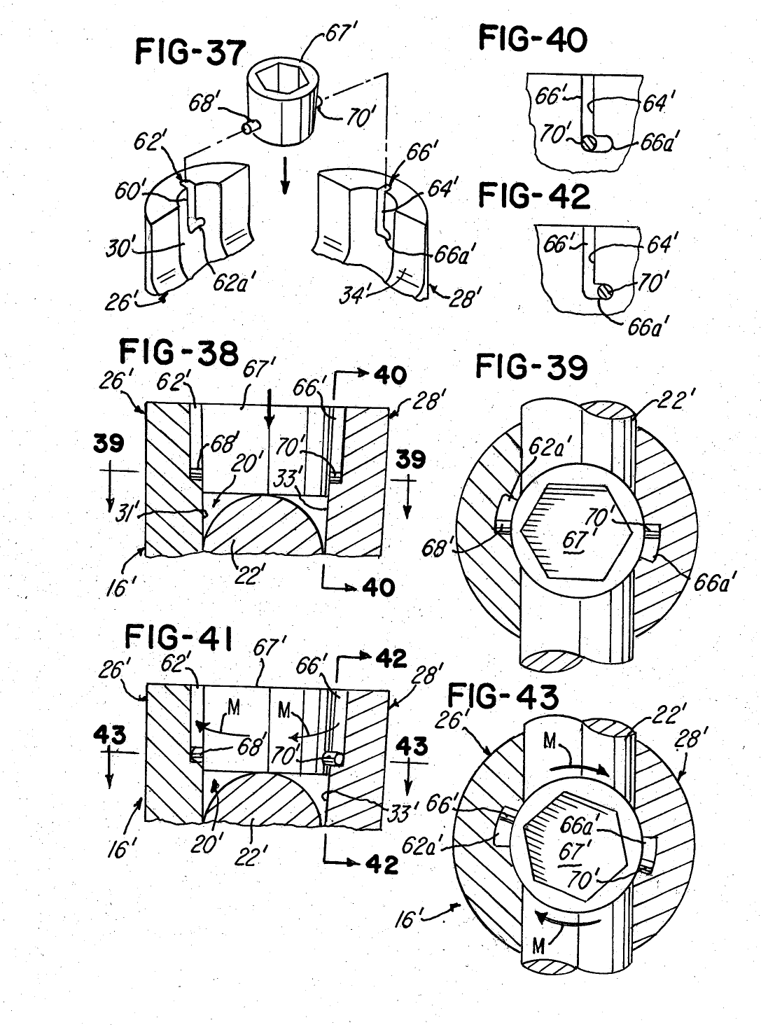

Referring now to FIGS. 37-43, another embodiment is shown. Like parts are identified with the same part number except that the parts in the embodiment of FIGS. 37-43 have a prime mark (“′”) added thereto. In this embodiment, a bayonet connection is provided for locking the cap 67′ to the receiver 16′. Notice that a wall 60′ is provided in rod-receiving wall 30′ and defines a generally L-shaped opening or channel 62′. A wall 64′ is provided in rod-receiving wall 34′ and defines a generally L-shaped channel or opening 66′. Notice that the cap 67′ comprises male projections 68′ and 70′ that are received in and cooperate with channels 62′ and 66′ to provide a bayonet connection. In this regard, notice that the cap 67′ is inserted into the rod-receiving area 20′ and in the direction of arrow L (FIGS. 37 and 38) until the projections, such as projection 70′ in FIG. 40, reaches a bottom surface 66a′ of wall 66′ (FIGS. 40, 42) as shown.

The male projections 68′ and 70′ are received in the female apertures or channels 62′ and 66′, respectively, and the cap 67′ is moved in the direction of arrow L (FIGS. 38 and 40) until the male projections 68′ and 70′ engage a bottom surface 62a′ and 66a′ (as viewed in FIG. 37). Thereafter, the cap 67′ is rotated clockwise (as viewed in FIG. 41) and the projections 68′ and 70′ are received at the end 62a′ and 66a′ of the channels 62′ and 66′, respectively. The bayonet connection prevents the cap 67′ from moving out of the receiver 16′, thereby locking the cap 67′ in the receiver 16′.

As with the threaded cap 24 in the embodiment shown in FIG. 1, notice that the cap 67′ in the embodiment of FIGS. 37-43 is also tapered or trapezoidal in cross-section so that as the cap 67′ is inserted into the receiver 16′, the first and second rod-receiving walls 30′ and 34′ are urged or forced away from each other which causes socket walls 32′ and 36′ to move toward polyaxial screw head 14′ similar to the embodiment described earlier herein relative to FIGS. 1-12. The movement of the cap 67′ into the receiver 16′ continues until a bottom 66a′ (FIG. 37) engages the rod 22′ and locks the rod 22′ in receiver 16′ and to the polyaxial screw head 14′ of the screw 12′.

Advantageously, the embodiment of FIGS. 37-43 provide a bayonet connection for locking the cap 67′ to the receiver 16′ and thereby locking the rod 22′ onto the polyaxial screw head 14′ of the screw 12′. FIGS. 38-40 illustrate the cap 67′ after it is received in the receiver 16′ and in the unlocked position, while FIGS. 41-43 illustrate the cap 67′ after it has been rotated in a clockwise direction (in a direction of arrow M in FIGS. 41 and 43) to thereby lock the cap 67′ to the receiver 16′. Again, it should be understood that the movement of the cap 67′ in the direction of arrow L (as viewed in FIG. 37) forces or urges the walls 31′ and 33′ away from each other which in turn causes the walls 32′ and 36′ defining the socket area 18′ to become closer together until they engage and lock against the polyaxial screw head 14′ of the screw 12′.

Referring now to FIGS. 44-47, another embodiment of the invention is shown. In this embodiment, like parts are identified with the same part numbers, except that a double prime mark (“″”) has been added to the part numbers in the embodiment of FIGS. 44-47. As mentioned earlier herein, receiver 16 may comprise a pivot connection or pivot joint, as illustrated in FIGS. 1-12. In this embodiment, the first and second receiver members 26 and 28 are integral formed or provided and comprise an integral pivot area 55″ comprising a resilient, elastic or deformable material that permits pivoting movement of the rod-receiving walls 30″ and 34″ relative to each other or about the pivot axis PA2 (FIG. 45).

In the embodiment being described, the resilient or deformable material may be defined by an elastomeric material, such as a metallic material (i.e. steel or titanium), a composite material, a polymer or a bio-compatible material. As with the embodiment described earlier herein relative to FIGS. 1-12, notice that when the threaded cap or screw 24″ is threadably received between the threads 30a″ and 34a″, the rod-receiving walls 30″ and 34″ are urged or moved away from each other. In response, the first and second socket walls 32″ and 36″ pivot or move toward each other. Thus, it should be understood that the receiver members 26″ and 28″ are integrally coupled and adapted to permit pivoting movement of the receiver members 26″ and 28″, respectively to permit bottom loading of the polyaxial screw head 14″ of the screw 12″ in a manner similar to that described earlier herein relative to FIGS. 1-12.

In the illustration being described, it should be understood that while the integral pivot area 55″ may be integrally formed between the receiver members 26″ and 28″ as shown, a multi-piece construction wherein a metal, elastomeric or other resilient or deformable material is secured between the receiver members 26″ and 28″ to permit the aforementioned pivoting connection or movement.

In the illustration being described, the receiver 16″ comprises a generally U-shaped wall 72″ (FIG. 46) that may comprise a notched-out area 74″. Likewise, a socket wall 76″ defines the socket area 18″ and may comprise a notched-out area 78″ as shown. In the illustration being described the notched-out areas 74″ and 78″ facilitate permitting the pivotal movement of the first receiver member 26″ and the second receiver member 28″ in order to permit the socket walls 32″ and 36″ that cooperate to define the socket area 18 to engage and lock against the polyaxial screw head 14″ of the screw 12″ after the rod 22″ is received in the rod-receiving area 20″ similar to the manner described earlier herein relative to FIGS. 9-12.

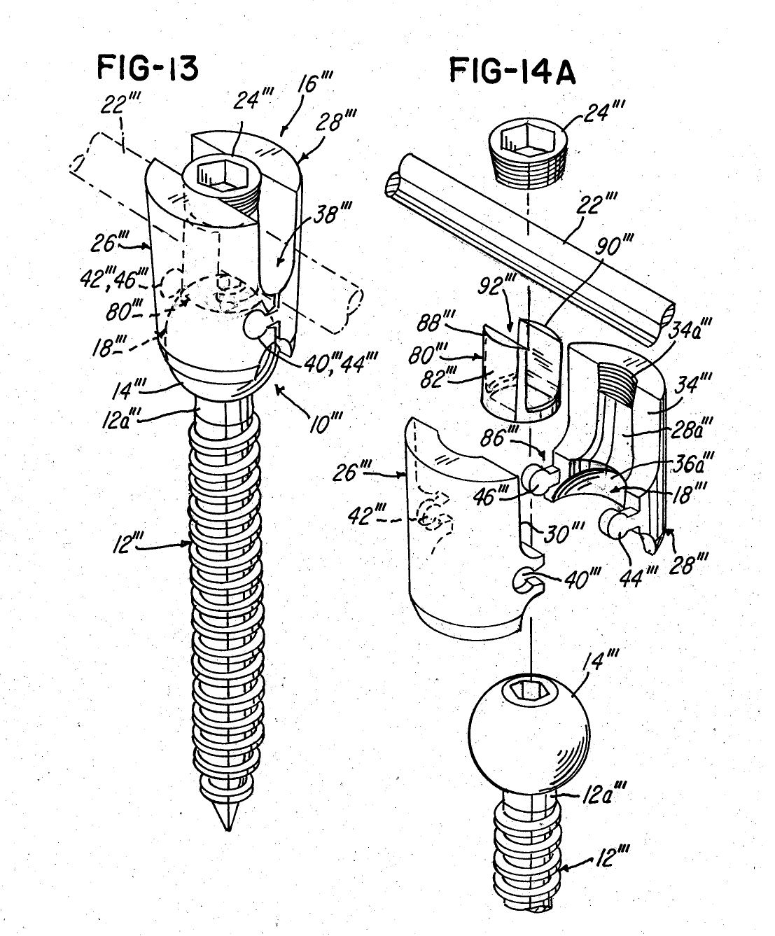

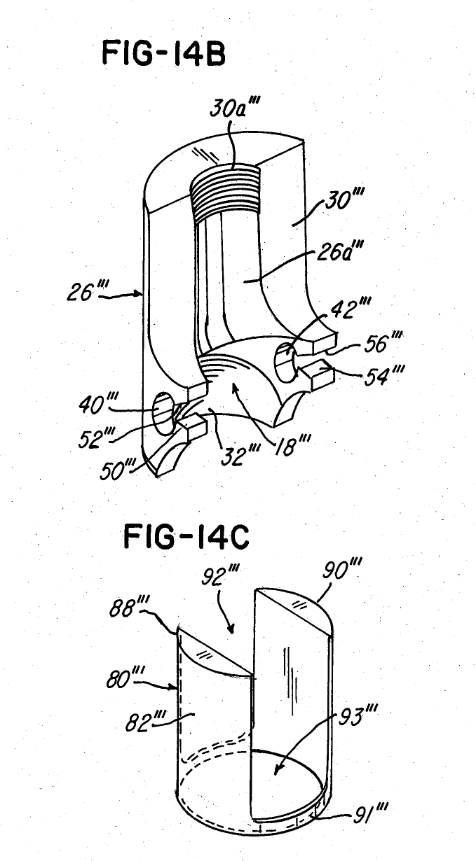

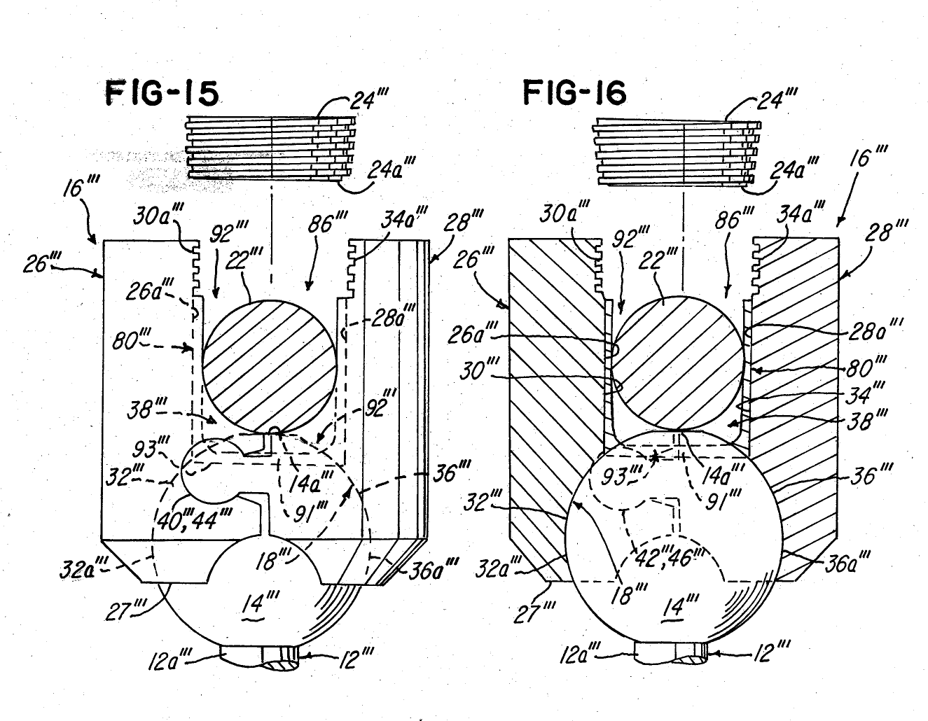

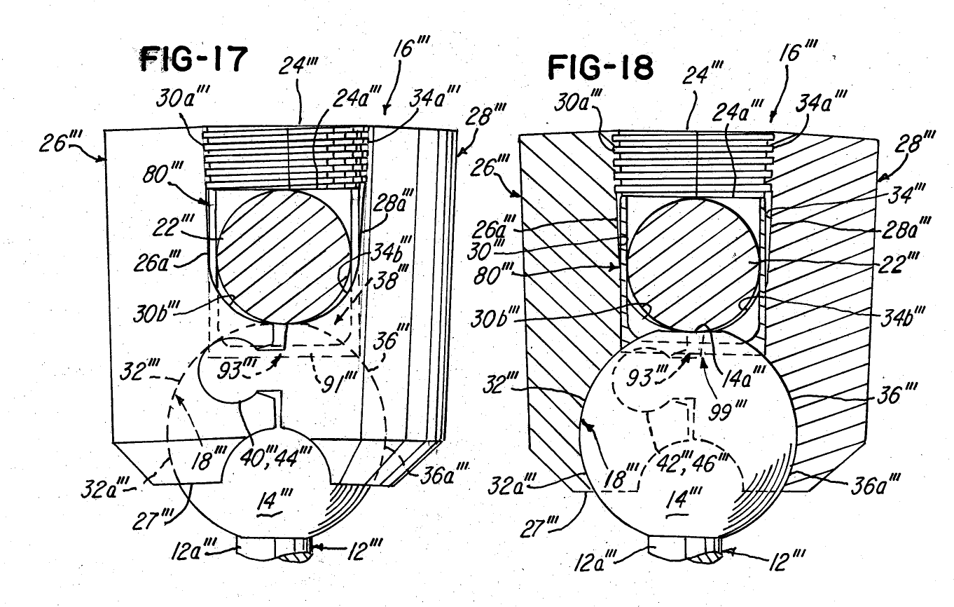

FIGS. 13-24 illustrate another embodiment, with like parts being identified with the same part number except that the part numbers in this embodiment have a triple prime (“′″”) mark added thereto. In this embodiment a guide sleeve 80′″ is used to facilitate guiding the rod 22′″ into the receiver 16′″. In this embodiment it has also been found that the guide sleeve 80′″ facilitates maintaining the rod-receiving walls 30′″ and 34′″ in an open or spread position (as shown in FIGS. 16 and 18) after the socket area 18′″ receives the polyaxial screw head 14′″ of the screw 12′″. This is particularly useful during assembly and transportation of the system 10′″. In this regard, note that the receiver members 26′″ and 28′″ of the receiver 16′″ may comprise an arcuate or machined area having a generally cylindrical or curved surface, area or wall 26a′″ (FIG. 14B) and curved surface, area or wall 28a′″ (FIG. 14A), respectively, having a curvature that generally complements or is adapted to complement a shape of an outer surface 82′″ of the guide sleeve 80′″. The surfaces or areas 26a′″ and 28a′″ cooperate to define a guide sleeve area 86′″ (FIGS. 14A, 15 and 16). Features of the receiver and guide sleeve illustrated in U.S. patent application Ser. No. 11/610,698, filed Dec. 14, 2006, which is assigned to the same assignee as the present invention and which is incorporated herein by reference and made a part hereof, may be incorporated and used with the invention described herein.

The guide sleeve 80′″ comprises a wall 91′″ defining an opening 93′″ (FIG. 14C). The guide sleeve 80′″ also comprises a first guide sleeve wall 88′″ and a second guide sleeve wall 90′″ that cooperate to define a guide sleeve rod-receiving area or generally U-shaped channel 92′″. Note that in this embodiment, the assembled receiver 16′″ comprises an opening 99′″ (FIG. 18) that provides communication among the guide sleeve area 86′″, rod receiving area or channel 38′″ and socket area 18′″ that permits at least a portion 14a′″ of polyaxial screw head 14′″ to extend through openings 93′″ (FIGS. 14C, 15-18) and 99′″ (FIG. 18) and into the rod-receiving area 38′″ and the guide sleeve rod-receiving area 92′″. In contrast, note that in the embodiment of FIGS. 1-12, the socket area 18 does not extend into the rod-receiving area 38, as illustrated in FIGS. 4 and 6. In the embodiment of FIGS. 13-24, however, the socket area 18′″ and first and second socket walls 32′″ and 36′″ are adapted and dimensioned such that the socket area 18′″ is in communication with the rod-receiving areas 38′″ and 92′″ so that at least a portion 14a′″ of the polyaxial screw head 14′″ may extend into both rod-receiving areas 38′″ and 92′″ after the socket area 18′″ receives the polyaxial screw head 14′″. Notice, for example, in FIGS. 15-18 that after the socket area 18′″ receives the polyaxial screw head 14′″, the portion 14a′″ (FIG. 16) of the polyaxial screw head 14′″ extends into the rod-receiving area 38′″. Notice also that the guide sleeve 80′″ opening 93′″ defined by an interior edge or wall 91 ′″ of guide sleeve 80′″ permits the portion 14a′″ of the 14′″ to extend into the guide sleeve rod-receiving area 92′″ and between the guide sleeve walls 88′″ and 90′″ that define the guide sleeve rod-receiving area 92′″.

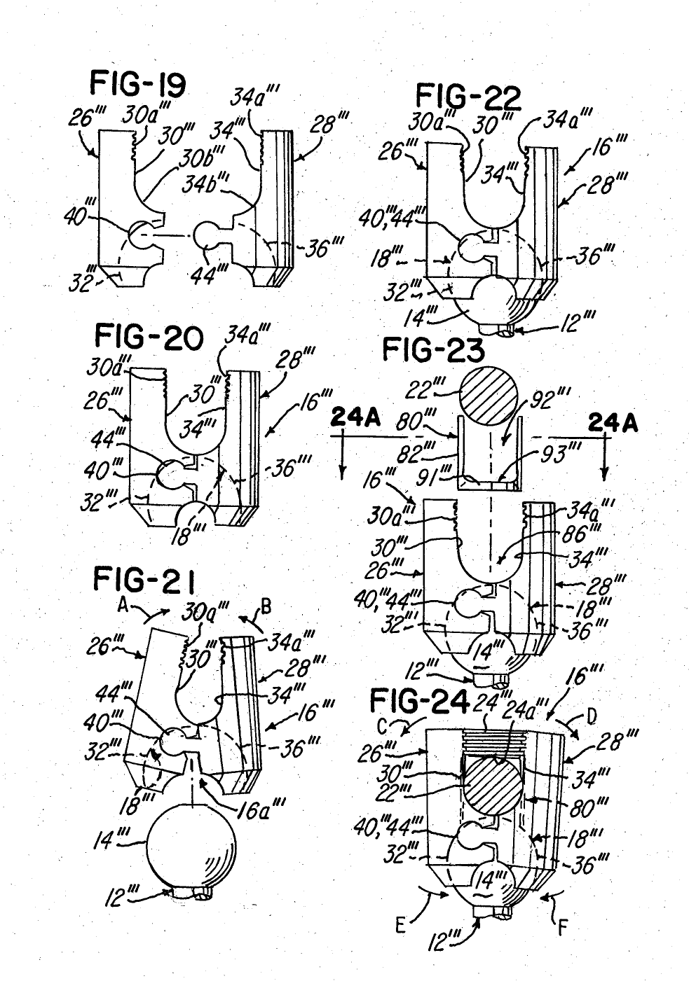

The first and second receiver members 26′″ and 28′″ are assembled as illustrated in FIGS. 19 and 20 in the manner described earlier herein relative to the embodiment shown in FIG. 1. The rod-receiving walls 30′″ and 34′″ are moved toward each other so that the polyaxial screw head 14′″ of the screw 12′″ can be bottom-fed into the socket area 18′″, as illustrated in FIGS. 21 and 22. Next, the rod-receiving walls 30′″ and 34′″ are urged away from each other and the guide sleeve 80′″ is inserted into the rod receiving area 38′″ which is an area between the two arcuate or curved surfaces 30b′″ (FIG. 19) and 34b′″. Notice that when the guide sleeve 80′″ is received in the area 86′″ between the rod-receiving walls 30′″ and 34′″, the walls are prevented from collapsing toward each other which in turn prevents the socket area from opening so that the polyaxial screw head 14′″ cannot become unlocked from the receiver 16′″.

Notice in FIG. 24A, the receiver member 26′″ comprises wall 26a′″ that is arcuate or curved and adapted to complement the shape of the guide sleeve 80′″. Likewise, second receiver member 28′″ comprises arcuate or curved wall 28a′″. The walls 26a′″ and 28a′″ cooperate to define an aperture for receiving the receiver member 26′″. The receiver member 26′″ also comprises wall 89′″ and second receiver member 28′″ comprises wall 91′″. The walls 89′″ and 91′″ cooperate to define an opening 93′″ of guide sleeve 80′″ so that a tool may be inserted through the receiver 16′″ to screw the screw 12′″ before the rod 22′″ is received in the rod-receiving area 38′″. After the guide sleeve 80′″ is received in the guide sleeve area 86′″, the rod 22′″ can be received in the guide sleeve 80′″ and in the rod-receiving channel.

Advantageously, this receiver 16′″ permits the portion 14a′″ to extend into both rod-receiving areas 38′″ and 92′″ so that when the threaded cap 24′″ is threadably received in the receiver 16′″, the bottom surface 24a′″ engages the rod 22′″ and urges it downward until it directly engages the polyaxial screw head 14′″ as illustrated in FIGS. 15-18 and 24. This causes the rod 22′″ to directly engage and lock against the polyaxial screw head 14′″.

As with the prior embodiments, the threaded cap 24′″ is generally trapezoidal in cross-section so that the rod-receiving walls 30′″ and 34′″ are moved or urged away from each other and thereby pivotally urge the first and second socket walls 32′″ and 36′″ toward each other in order to lock the receiver 16′″ directly to the polyaxial screw head 14′″.

During use, the receiver 16′″ is assembled as illustrated in FIGS. 19-20, the polyaxial screw head 14′″ is bottom-fed into the socket area 18′″ in the manner shown in FIGS. 21-22. Thereafter, the guide sleeve 80′″ is inserted between the walls 26a″ and 28a′″ and into the area 86′″ (FIG. 15) between the walls 26a′″ and 28a′″. The rod 22′″ is then received in the rod-receiving areas 38′″ and 92′″ and then the threaded cap 24′″ is rotatably threaded into the threads 30a′″ and 34a′″. As the threaded cap 24′″ is screwed into the receiver 16′″ it urges the socket walls 32′″ and 36′″ toward each other until they engage and lock against the polyaxial screw head 14′″. The bottom surface 24a′″ of the threaded cap 24′″ ultimately engages the surface 22a′″ of the rod 22′″ and urges it or forces it downward until the rod 22′″ engages the portion 14a′″ of the polyaxial screw head 14′″, as illustrated in FIGS. 15-18. Thus, the threaded cap 24′″ is rotated until the rod 22′″ engages and becomes locked against the polyaxial screw head 14′″ and the socket walls 32′″ and 36′″ engage and lock against the polyaxial screw head 14′″.

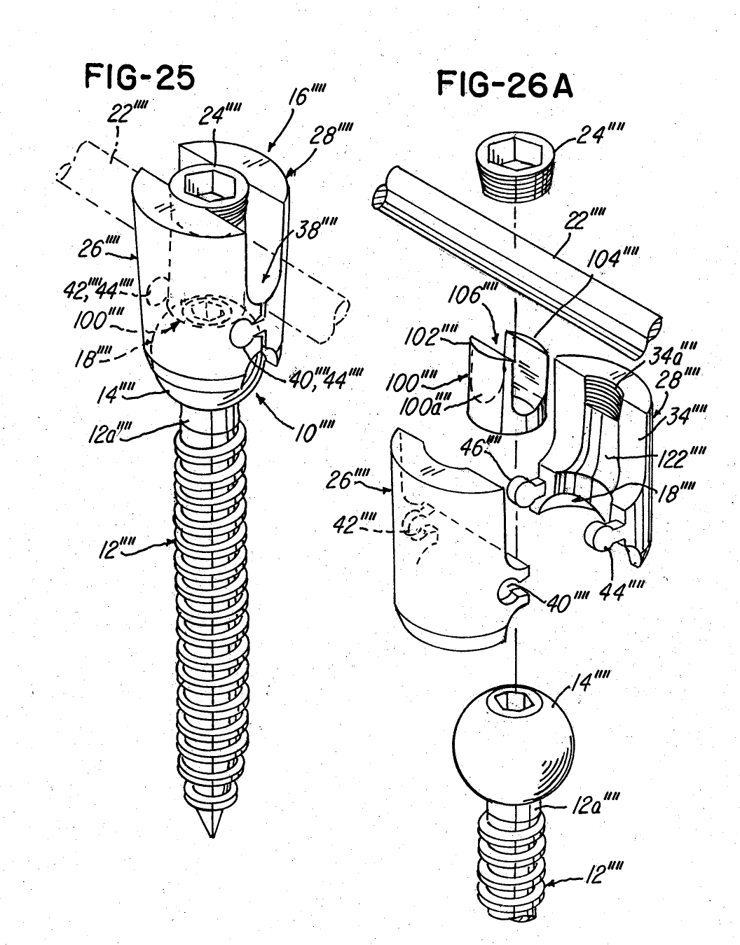

It should be understood that the embodiment of FIGS. 13-24 provides a guide sleeve 80′″ that functions to provide a guide for the rod 22′″, but the sleeve 80′″ does not provide a compressive locking force against the portion 14a′″ for locking the rod 22″ to the polyaxial screw head 14′″ of the screw 12′″. In contrast, another embodiment is illustrated in FIGS. 25-36 wherein a compression member 100′″ (as shown, for example, in FIG. 35) is used with receiver 16′″. In this embodiment, the compression member 100′″ has a first wall 102′″ and a second wall 104′″ and a rod-receiving area or channel 106′″ between the first and second walls 102′″ and 104′″.

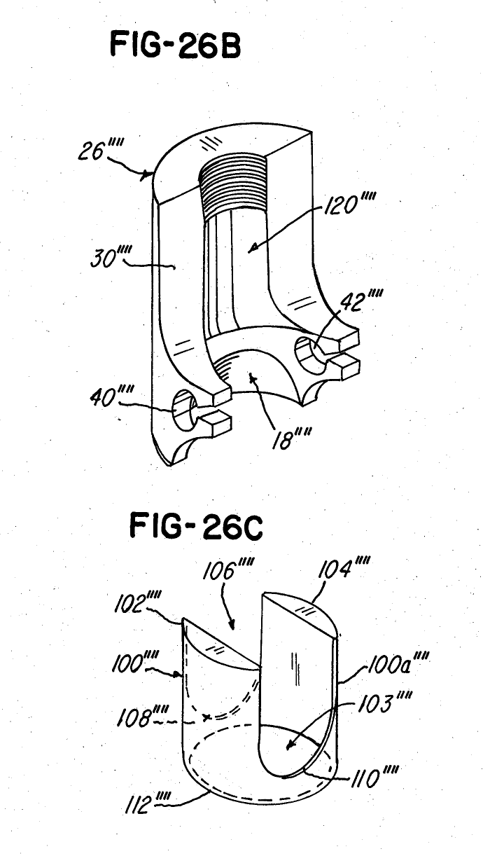

Those parts that are the same or similar to the parts illustrated earlier herein are identified with the same part numbers except a quadruple prime mark (“″″”) has been added to the parts in the embodiment shown in FIGS. 25-36. The compression member 100″″ has a pair of generally opposed walls 102″″ and 104″″ that cooperate to define a channel 106″″ that receives rod 22″″. The compression member 100″″ further comprises generally U-shaped walls 108″″ and 110″″ (FIG. 26) and a bottom edge or surface 112″″ that may be tapered or curved.

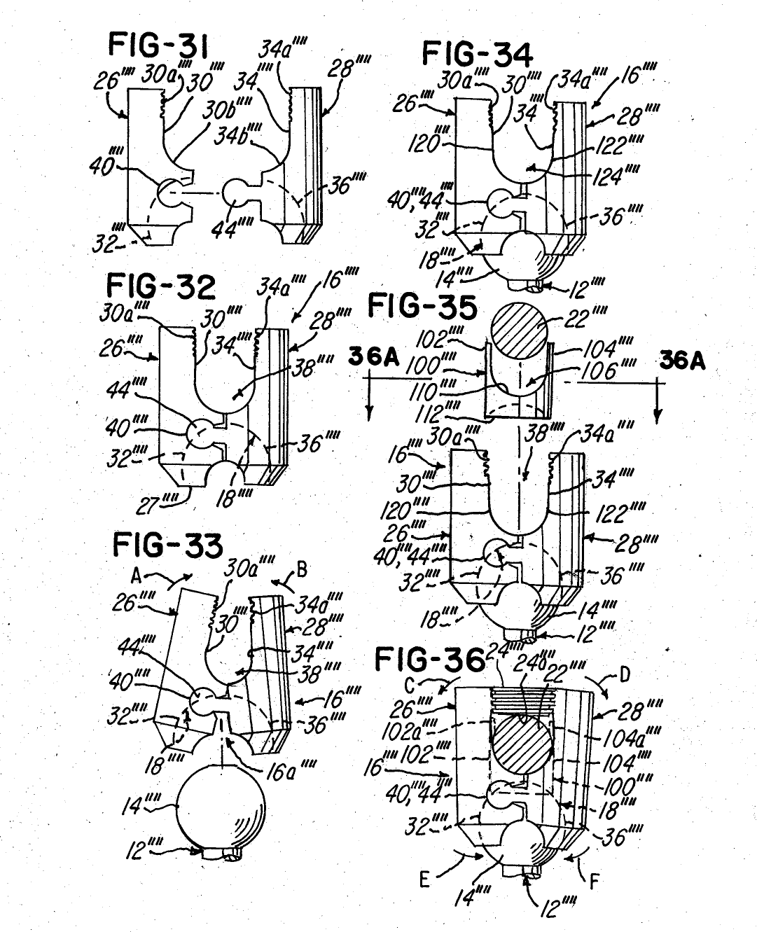

In this embodiment, the receiver members 26″″ and 28″″ are assembled as illustrated in FIGS. 31 and 32 and then pivoted as illustrated in FIG. 33 so that the polyaxial screw head 14″″ can be inserted into the socket area 18″″. Next, the compression member 100″″ is inserted between the rod-receiving walls 30″″ and 34″″ as illustrated in FIGS. 35 and 36. In this regard, notice that the rod-receiving walls 30′″ and 34′″ have an arcuate or curved or generally U-shaped surface 120″″ and 122″″, respectively, (FIG. 28), similar to surfaces 26a″″ and 28a″″ in the previous embodiment. The surfaces 120″″ and 122″″ generally complement or are adapted to complement the outer surface 100a″″ of the compression member 100″″. Note in FIG. 36A the surfaces 120″″ and 122″″ cooperate to define an aperture 107″″ that is in communication with the aperture 103″″ so that a tool may be inserted into the receiver 16″″ to screw the screw 12″″. Thus, similar to the embodiment illustrated earlier herein relative to FIGS. 13-24, the receiver 16″″ in this embodiment comprises the rod-receiving area 38″″ and also a compression member 100″″ receiving area 124″″, which is the area between the curved surfaces 120″″ and 122″″.

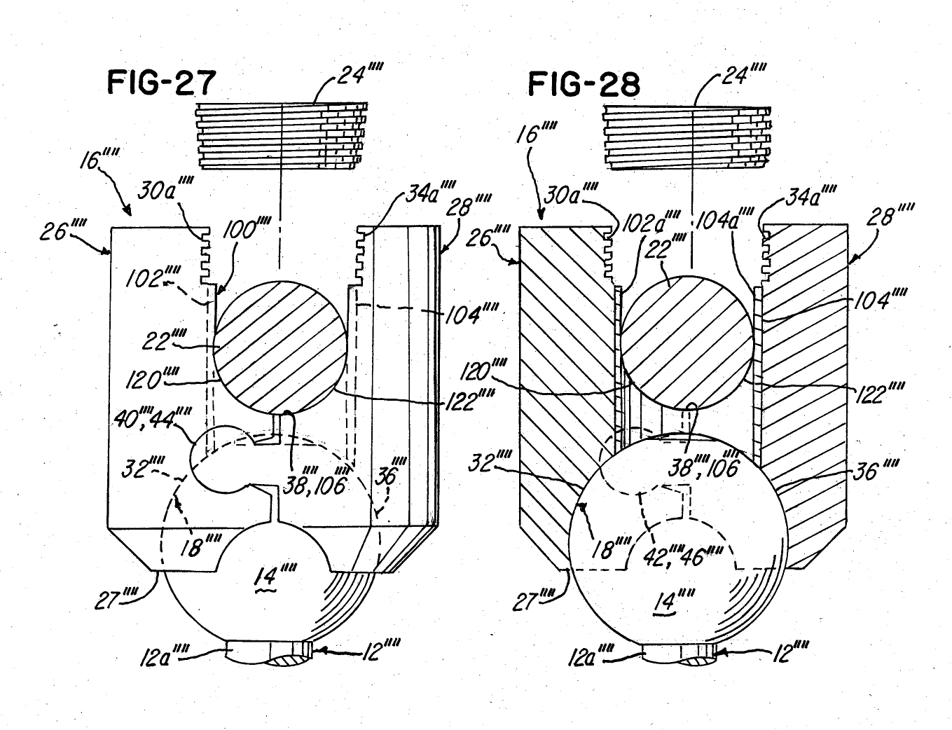

After the compression member 100″″ is received in the compression member 100″″ receiving area 124″″ and in the receiver 16″″, the rod-receiving areas or channels 38″″ and 106″″ are aligned and the rod 22″″ is received therein, as illustrated in FIGS. 35-36. Notice that a portion of the rod 22″″ extends above the ends 102a″″ and 104a″″ (FIG. 28) of the walls 102″″ and 104″″. As shown in FIGS. 27-30, the threaded cap 24″″ is rotatably received between the threads 30a″″ and 34a″″ and is screwed into the receiver 16″″ until the bottom 24a″″ (FIG. 24A) engages the rod 22″″. As the threaded cap 24″″ is screwed into the receiver 16″″, the receiver members 26″″ and 28″″ pivot relative to each other so that the rod-receiving walls 30″″ and 34″″ move away from each other while the first and second socket walls 32″″ and 36″″ move toward each other until they lock against the polyaxial screw head 14″″ of screw 12″″. As the threaded cap 24″″ continues to rotate, the rod 22″″ is driven toward and engages the generally U-shaped surfaces 120″″ and 122″″ (FIG. 26) and forces the compression member 100″″ downward (as viewed in FIGS. 35 and 36) until the rod 22″″ also engages the polyaxial screw head 14″″ to provide further locking of the rod 22″″ to the polyaxial screw head 14″″.

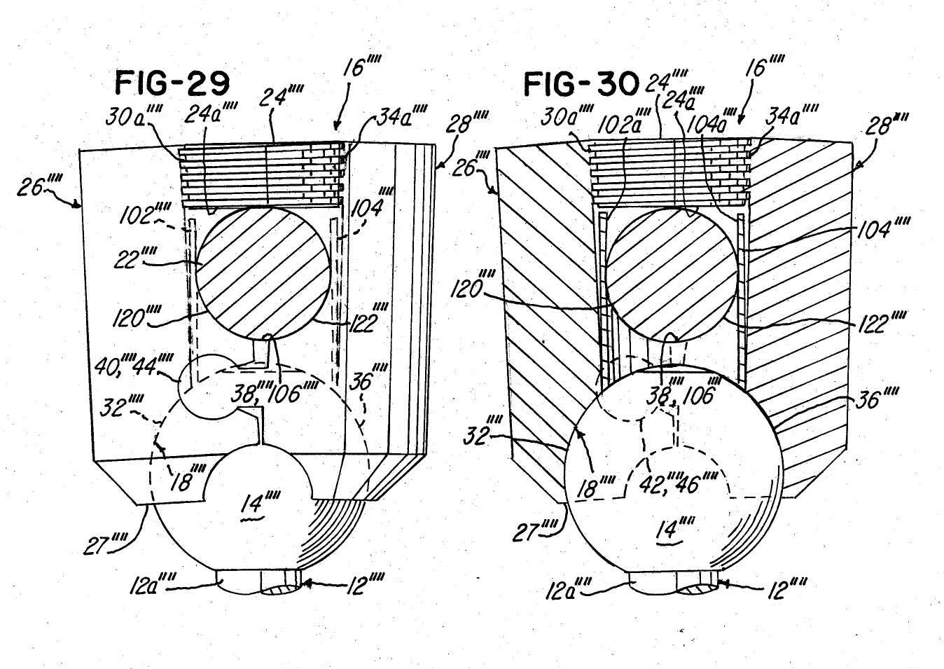

Thus, in the embodiment being described relative to FIGS. 25-36, multiple locking is provided. First socket walls 32″″ and 36″″ lock onto the polyaxial screw head 14″″, and the bottom surface 112″″ of compression member 100″″ engages and locks onto the polyaxial screw head 14″″ after the threaded cap 24″″ is threadably received in the receiver 16″″ as illustrated in FIGS. 29, 30 and 36. In this embodiment, therefore, multiple locking is provided to provide enhanced locking of the rod 22″″ to the polyaxial screw head 14″″.

Advantageously, the polyaxial screw system 10 and method described herein relative to the various embodiments shown in FIGS. 1-47 provides a means, apparatus and methodology for locking a rod onto a polyaxial screw by using a receiver wherein the polyaxial screw head 14 of the screw 12 can be bottom-loaded and the receiver is adapted to permit a dimension of the socket area to change so that the receiver 16 can lock onto the polyaxial screw head 14 of the screw 12. In the illustrations being described, the receiver 16 provides pivoting or teetering movement so as to enable at least one or a plurality of socket walls 32 and 36 to pivot toward and engage the polyaxial screw head 14 of the screw 12. The socket walls 32 and 36 exert a generally lateral force on a bottom portion of the polyaxial screw head 14 and force it upward into the socket area 18. It is anticipated that the various parts will be assembled so that the polyaxial screw head 14 is received in the socket area 18 prior to use. During use, the screw 12 will be screwed into a bone, such as a vertebra, and the receiver 16 will be pivoted or angulated to a desired position. The rod 22 will be situated in the receiver 16 and then the threaded cap 24 screwed in the receiver 16 in order to lock the rod to the polyaxial screw head 14 in the manner described herein.

While the various embodiments show either a threaded cap or a bayonet (FIG. 37) type cap, it should be understood that other means for forcing the rod-receiving walls 30 and 34 apart may be provided. For example, the areas 30b and 34b of rod-receiving walls 30 and 34, respectively, may be provided with ridges or detents (not shown), rather than threads, so that a wedge cap (not shown) may be situated between the rod-receiving walls 30 and 34. Although not shown, external devices for forcing the socket walls 32 and 36 toward each other may also be provided without departing from the true spirit and scope of the invention which is to provide a receiver 16 that has a socket area 18 that is adapted to change to lock against the polyaxial screw head 14 of the screw 12.

In the illustrations, the various receivers, such as receiver 16 in the first illustration, and various components may be made from a metallic material (i.e. steel or titanium), a composite material, a polymer or a bio-compatible material.

While the apparatus and method described constitute preferred embodiments of this invention, it is to be understood that the invention is not limited to this precise apparatus and method and that changes may be made in either without departing from the scope of the invention, which is defined in the appended claims.

What is claimed is:

1. A polyaxial screw system comprising:
a receiver comprising a socket for receiving a head of a polyaxial screw and a rod-receiving area for receiving a rod;
said receiver having a pivot area to permit a dimension of said socket to be changed in order to apply a locking force onto said head of said polyaxial screw.

2. The polyaxial screw system as recited in claim 1 wherein said pivot area is between said rod-receiving area and said socket.

3. The polyaxial screw system as recited in claim 1 wherein said receiver comprises a resilient or deformable material at said pivot area.

4. The polyaxial screw system as recited in claim 3 wherein said resilient or deformable material comprises an elastomeric material, such as a metallic material (i.e. steel or titanium), a composite material, a polymer or a bio-compatible material.

5. The polyaxial screw system as recited in claim 1 wherein said pivot area comprises a pivot joint.

6. The polyaxial screw system as recited in claim 1 wherein said head of said polyaxial screw is loaded into said socket through a bottom of said receiver.

7. The polyaxial screw system as recited in claim 6 wherein said bottom of said receiver and said head of said polyaxial screw are each adapted to permit angulation of said polyaxial screw.

8. The polyaxial screw system as recited in claim 5 wherein said pivot joint comprises at least one male projection and at least one female receiving area for receiving said at least one male projection that cooperate to define said pivot joint.

9. The polyaxial screw system as recited in claim 1 wherein said receiver comprises a resilient or deformable material situated at said pivot area comprises at least one male pivot connector and at least one female pivot connector that cooperate to define said pivot joint.

10. The polyaxial screw system as recited in claim 1 wherein said receiver comprises a first member and a second member that are pivotally connected at said pivot area, said first member and said second member being adapted so that they cooperate to define said rod-receiving area and said socket after said first and second members are pivotally connected together.

11. The polyaxial screw system as recited in claim 1 wherein said system further comprises:
a cap adapted to be received in said rod-receiving area,
said cap causing at least a portion of said receiver to pivot at said pivot area to change said dimension of said socket in order to apply a locking force onto said head of said polyaxial screw in response to said cap being situated in said rod-receiving area.

12. The polyaxial screw system as recited in claim 11 wherein said cap is dimensioned to apply a lateral force against at least one rod-receiving wall associated with said rod-receiving area of said receiver.

13. The polyaxial screw system as recited in claim 12 wherein said lateral force causes said at least one rod-receiving wall to be urged away from an axis of said receiver.

14. The polyaxial screw system as recited in claim 12 said receiver is adapted to decrease said dimension of said socket in response to said lateral force.

15. The polyaxial screw system as recited in claim 14 wherein said receiver comprises a first socket wall and a generally opposed second socket wall;
said cap being adapted to cause a distance between said first and second socket walls to become smaller to apply said locking force to said head of said polyaxial screw when said cap is received in said rod-receiving area.

16. The polyaxial screw system as recited in claim 15 wherein said cap is threaded.

17. The polyaxial screw system as recited in claim 15 wherein said cap is trapezoidal in cross section.

18. The polyaxial screw system as recited in claim 15 wherein said receiver comprises at least one bayonet channel and said cap comprises at least one male projection that can be received in said at least one bayonet channel to provide a bayonet connection.

19. The polyaxial screw system as recited in claim 1 wherein said system further comprises a cap adapted to cause said socket to apply said locking force to lock said receiver onto said head of said polyaxial screw and to also lock said rod in said receiver in response to said cap being received in said rod-receiving area.

20. The polyaxial screw system as recited in claim 15 wherein said cap is at least one of threaded or trapezoidal in cross section.

21. The polyaxial screw system as recited in claim 15 wherein said locking force is in a generally radial direction relative to an axis of said receiver.

22. The polyaxial screw system as recited in claim 1 wherein said socket is defined by a first socket wall and a second socket wall, at least one of said first or second socket walls move toward each other to apply said locking force.

23. The polyaxial screw system as recited in claim 22 wherein both first and second socket walls move toward each other to apply said locking force.

24. The polyaxial screw system as recited in claim 1 wherein said system further comprises a guide sleeve adapted to be received in said receiver.

25. The polyaxial screw system as recited in claim 1 wherein said system comprises a compression member for providing a compression locking force against said head of said polyaxial screw and adapted to be received in said receiver.

26. A bone fixation system comprising:
a receiver comprising a socket defining a socket area, said socket comprising at least one socket wall, said socket being adapted to receive a head of a polyaxial screw; a rod-receiving area defined by at least one rod-receiving wall, said rod-receiving area being adapted to receive a rod, and a pivot area between said rod-receiving area and said socket area;
said pivot area permitting a dimension of said rod-receiving area to increase and substantially simultaneously being adapted to permit a dimension of said socket area to decrease so that said at least one socket wall may become locked to said head of said polyaxial screw in response to movement of said at least one rod-receiving wall.

27. The bone fixation system as recited in claim 26 wherein said movement of said at least one rod-receiving wall is away from an axis of said receiver.

28. The bone fixation system as recited in claim 26 wherein said socket is defined by a plurality of socket walls that cooperate to define said socket, said plurality of socket walls not being integral or continuous.

29. The bone fixation system as recited in claim 26 wherein said pivot area comprises a pivot joint.

30. The bone fixation system as recited in claim 26 wherein said receiver comprises a material at said pivot joint that is adapted to permit said at least one socket wall and said at least one rod-receiving wall to pivot about a pivot axis.

31. The bone fixation system as recited in claim 30 wherein said pivot axis is generally orthogonal to an axis of said receiver.

32. The bone fixation system as recited in claim 30 wherein said resilient or deformable material comprises an elastomeric material, such as a metallic material (i.e. steel or titanium), a composite material, a polymer or a bio-compatible material.

33. The bone fixation system as recited in claim 26 wherein said pivot area is between said rod-receiving area and said socket area.

34. The bone fixation system as recited in claim 26 wherein said head of said polyaxial screw is loaded into said socket through a bottom of said receiver.

35. The bone fixation system as recited in claim 34 wherein said bottom of said receiver and said head of said polyaxial screw are each adapted to permit angulation of said polyaxial screw.

36. The bone fixation system as recited in claim 29 wherein said receiver comprises a first wall and generally opposed second wall and a pivot joint pivotally coupling said first and second walls, said first wall comprises at least one male pivot connector and at least one female receiving area for receiving at least one male pivot connector that cooperate to define said pivot joint.

37. The bone fixation system as recited in claim 26 wherein said receiver comprises a first member and a second member that are pivotally connected at said pivot area, said first member and said second member being adapted to define said rod-receiving area and said socket area after said first and second members are pivotally connected together.

38. The bone fixation system as recited in claim 26, wherein said bone fixation system comprises a cap adapted to cause said receiver to pivot at said pivot area to change said dimension of said socket in order to apply a locking force onto said head of said polyaxial screw in response to said cap being situated in said rod-receiving area.

39. The bone fixation system as recited in claim 26 wherein said cap is dimensioned to apply a lateral force against at least one rod-receiving wall associated with said rod-receiving area of said receiver.

40. The bone fixation system as recited in claim 39 wherein said lateral force is away from an axis of said receiver.

41. The bone fixation system as recited in claim 39 said receiver is adapted to decrease said dimension of said socket in response to said lateral force.

42. The bone fixation system as recited in claim 26 wherein said receiver comprises a first socket wall and a generally opposed second socket wall;
said bone fixation system further comprising a cap adapted to cause said first and second socket walls to move closer together to apply said locking force to said head of said polyaxial screw when said cap is received in said rod-receiving area.

43. The bone fixation system as recited in claim 26 wherein said cap is trapezoidal in cross section.

44. The bone fixation system as recited in claim 42 wherein said cap is adapted to adapted to cause said socket to clamp onto said head of said polyaxial screw and substantially simultaneously lock said rod into said rod-receiving area.

45. The bone fixation system as recited in claim 42 wherein said cap is a threaded cap and trapezoidal in cross section.

46. The bone fixation system as recited in claim 42 wherein said at least one socket wall moves in a generally radial direction relative to an axis of said receiver to cause said socket to lock onto said head of said polyaxial screw.

47. The bone fixation system as recited in claim 26 wherein said socket is defined by a plurality of walls that cooperate to define said socket, but that are not integral.

48. The bone fixation system as recited in claim 26 wherein said system further comprises a guide sleeve adapted to be received in said receiver.

49. The bone fixation system as recited in claim 26 wherein said system comprises a compression member for providing a compression locking force against said head of said polyaxial screw and adapted to be received in said receiver.

50. The bone fixation system as recited in claim 26 wherein said receiver comprises at least one bayonet channel and said cap comprises at least one male projection that can be received in said at least one bayonet channel to provide a bayonet connection.

51. A receiver comprising:
a first rod-receiving wall and generally opposed second rod-receiving wall, said first and second rod-receiving walls cooperating to define a rod-receiving area for receiving a rod;
a first socket wall and a generally opposed second socket wall, said first and second socket walls cooperating to define a socket area for receiving a screw head of a screw; and
an intermediate area between said rod-receiving area and said socket area for permitting said first and second socket walls to move toward and away from each other in response to movement of at least one of said first or second rod-receiving walls.

52. The receiver as recited in claim 51 wherein each of said first and second rod-receiving walls comprise threads that cooperate to define a threaded aperture adapted to receive a threaded cap;
said threaded cap causing said first and second socket walls to become closer together when said cap is screwed into said threaded aperture.

53. The receiver as recited in claim 51 wherein said first rod-receiving wall is integral with said first socket wall to define a first portion of said receiver, said second rod-receiving wall is integral with said second socket wall to define a second portion of said receiver, said intermediate area being situated between said first and second portions.

54. The receiver as recited in claim 52 wherein said first and second rod-receiving walls are urged away from an axis of said receiver and said first and second socket walls being urged toward said axis when said threaded cap is screwed into said threaded aperture.

55. The receiver as recited in claim 53 wherein said intermediate area integrally couples said first portion to said second portion.

56. The receiver as recited in claim 53 wherein said intermediate area comprises a flexible or elastomeric metal or polymer material.

57. The receiver as recited in claim 53 wherein said intermediate area comprises a pivot joint for pivotally coupling.

58. The receiver as recited in claim 57 wherein said pivot joint comprises a first hinge member situated between said first rod-receiving wall and said first socket wall and a second hinge member situated between said second rod-receiving wall and said second socket wall, said first and second hinge members pivotally mating to enable said first and second socket walls to pivot.

59. The receiver as recited in claim 58 wherein said first and second hinge members are integral with said receiver.

60. The receiver as recited in claim 59 wherein said first and second hinge members each comprise a male member and a female opening that receives said male member to enable said first and second socket walls to pivot relative to each other.

61. A method for securing a polyaxial screw to a rod, said method comprising the steps of:
providing a receiver comprising a socket area for receiving a head of a polyaxial screw and a rod-receiving area for receiving a rod; and
enabling said receiver to pivot or flex at an intermediate area so that when a dimension of said rod-receiving area gets larger, a dimension of said socket area substantially simultaneously gets smaller to clamp against a screw head of said polyaxial screw.

62. The method as recited in claim 61 wherein said method further comprises the step of:
locating said intermediate area between said rod-receiving area and said socket.

63. The method as recited in claim 61 wherein said method further comprises the step of:
using a resilient or deformable material at said intermediate area.

64. The method as recited in claim 63 wherein said resilient or deformable material comprises an elastomeric material, such as a metallic material (i.e. steel or titanium), a composite material, a polymer or a bio-compatible material.

65. The method as recited in claim 61 wherein said method further comprises the step of:
pivotally coupling a first wall to a second wall, said first and second walls defining said socket area.

66. The method as recited in claim 61 wherein said method further comprises the steps of:
bottom loading said screw head into said socket area through a bottom of said receiver.

67. The method as recited in claim 61 wherein said method further comprises the steps of:
inserting a guide sleeve or compression member into said receiver after a screw head is received in said socket area.

68. The method as recited in claim 61 wherein said method further comprises the step of:
adapting a bottom of said receiver to permit angulation of said polyaxial screw.

69. The method as recited in claim 65 wherein said pivotally coupling step further comprises the step of:
pivotally coupling at least one male pivot connector and at least one female pivot connector together to define said pivot joint.

70. The method as recited in claim 61 wherein said method further comprises the step of:
providing a cap adapted to be received in said rod-receiving area,
said cap causing said receiver to pivot at said pivot area to change said dimension of said socket area in order to apply a locking force onto said head of said polyaxial screw in response to said cap being situated in said rod-receiving area.

71. The method as recited in claim 70 wherein said cap is dimensioned to apply a lateral force against at least one rod-receiving wall associated with said rod-receiving area of said receiver, thereby causing said at least one rod-receiving wall to move away from an axis of said receiver.

72. The method as recited in claim 70 wherein said cap is threaded.

73. The method as recited in claim 61 wherein said method further comprises the step of:
providing a first receiver half and a second receiver half;
pivotally coupling said first receiver half to said second receiver half;
said first and second receiver halves cooperating to define said socket area and said rod-receiving area and being adapted to cause at least one dimension of said socket area to change in response to pivoting movement between said first and second receiver halves.

74. A method of assembling a receiver comprising the steps of:
providing a receiver having a pivot area;
actuating said receiver to increase a dimension of a socket area;
inserting a screw head into the socket area; and
actuating said receiver to capture said screw head in said socket area.

75. The method as recited in claim 74 wherein said actuating step comprises the step of:
pivoting a wall of said receiver to perform said actuating step.

76. The method as recited in claim 74 wherein said method further comprises the step of:
inserting either a compression member or guide sleeve into said receiver after said inserting step.

77. The method as recited in claim 75 wherein said method further comprises the step of:
inserting either a compression member or guide sleeve into said receiver after said inserting step.
